(19) United States
(12) Patent Application Publication
(10) Pub. No.: US 2015/0257924 A1
(43) Pub. Date: Sep. 17, 2015

(54) Male Urinary Incontinence Device

(71) Applicant: Linda N. Siegel, Sarasota, FL (US)

(72) Inventor: Linda N. Siegel, Sarasota, FL (US)

(21) Appl. No.: 14/644,947

(22) Filed: Mar. 11, 2015

Related U.S. Application Data
(60) Provisional application No. 61/951,293, filed on Mar. 11, 2014.

(51) Int. Cl. A61F 5/453

(52) U.S. Cl. CPC A61F 5/453

(57) ABSTRACT

A device is embodied and broadly described herein which provides the user with a breathable comfortable device for urinary incontinence and leakage. The first preferred embodiment of the invention includes a generally collapsible cylindrically-shaped body having an open end and a closed end made of a flexible and breathable netting material that can expand and contract with the natural expanding and contracting of the penis. A sheet is attached to the inside of the closed end, having a layer of moisture absorbent material, a layer of waterproof material to prevent leakage onto the users clothing, and being attached to the inside of the closed end of the body by an adhesive.

An additional embodiment of the invention includes sheet having a non-permanent adhesive material bonding the sheet to the body, allowing the user to remove the sheet from the netting and replace it with a new sheet.

### BACKGROUND OF THE INVENTION

1. Field of the Invention

The present invention generally relates to the field of medical apparatus for male urinary incontinence, specifically, a urinary incontinence device system by means of a breathable mesh body having an open end for insertion of the user's penis and a sheet with absorbent layer and water-resistant material layer on the closed end, the securing of which being effected in a comfortable, reliable, hygienic, sanitary, convenient, discreet, and economical manner.

2. Description of the Prior Art

The use of wearable systems and their configurations for containing urinary leakage are known in the prior art. More specifically, wearable devices used for male urinary incontinence, designs and configurations previously devised to absorb leakage or dribbling through known methods and apparatus are known to consist of familiar and obvious structural configurations. Several designs in the prior art exist, for example: U.S. Pat. No. 6,248,096 issued to Dwork, Paul et. al., for male urinary incontinence device having expandable flutes; U.S. Pat. No. 3,742,953 issued to Lee for disposable male urinary incontinence device; U.S. Pat. No. 8,277,426 issued to Wilcox et. al. for male urinary incontinence device; U.S. Pat. No. 5,415,179 issued to Mendoza for male urinary incontinence device; and U.S. Pat. No. D589,610 issued to Dubose for male urinary incontinence device.

While these devices fulfill their respective objectives and requirements, they do not present a urinary incontinence device system by means of a breathable mesh body having an open end for insertion of the user's penis and a sheet with absorbent layer and water-resistant material layer on the closed end, the securing of which being effected in a comfortable, reliable, hygienic, sanitary, convenient, discreet, and economical manner.

In this respect, the male urinary incontinence system according to the present invention substantially departs from the conventional concepts and designs of the prior art, and in doing so provides an apparatus primarily developed for the purpose of discretion, comfort, ease of use, maintaining the user's confidence and self esteem by not being burdensome, and by being economical in both the manufacturing and for the consumer.

### SUMMARY OF THE INVENTION

The present invention addresses the problem of minor urinary leakage or dribbling of urine as a result of urinary incontinence by providing a easy to use device that maintains superior in comfort while being worn throughout the day and will absorb accidental urine leakage. The present invention is comfortable to wear, easy to apply and remove, gives the user confidence, and is not intimidating as other prior existing incontinence devices such as clamps, leg bags, and urine collection devices. Also, it is not embarrassing like an adult diaper, pad or special underwear device, and it may be worn underneath traditional underwear.

To achieve the foregoing utility in accordance with the purpose of the invention, a device is embodied and broadly described herein which provides the user with a breathable comfortable device for minor urinary incontinence and dribbling. The first preferred embodiment of the invention includes a generally collapsible cylindrically-shaped body having an open end and a closed end made of a flexible and breathable mesh netting material that can expand and contract with the natural expanding and contracting of the penis. The rim of the open end of the body has a soft elastic band allowing the invention to gently and comfortably grip the skin at the base of the penis, preventing the device from falling off. A sheet is attached to the inside of the closed end of the body portion and rests against the end of the user's penis when worn to absorb urinary dribbling. The sheet has a layer of moisture absorbent material and a layer of waterproof material to prevent leakage onto the user's clothing, and is affixed to the inside of the closed end of the body portion by a permanent adhesive.

An additional embodiment of the invention includes a sheet having a non-permanent adhesive material bonding the sheet to the inside of the body, allowing the user to remove the sheet from the inside of the body portion and replace it with a new sheet.

It is to be understood that the present invention is not limited in its application to the details of construction and to the arrangements of the components set forth in this description or in the drawings. The invention may have other embodiments and various executions. It is also to be understood that the phraseology and terminology used herein should not be regarded as limiting. Those skilled in the art will appreciate that the conception upon which this disclosure is based may be utilized as a basis for designing of other similar devices, systems, or methods, for carrying out the several purposes of the present invention. Therefore, the claims herein should be regarded as including such equivalent constructions to the extent that they do not depart from the scope of the present invention.

### BRIEF DESCRIPTION OF THE DRAWINGS

Having generally described the various embodiments of the invention, reference will now be made to the accompanying drawings, which are not necessarily drawn to scale, and wherein:

FIG. 1 is a side view of the device in accordance with an embodiment of the invention.

FIG. 2 is a top view of the device in accordance with an embodiment of the invention.

### DETAILED DESCRIPTION OF THE VARIOUS EMBODIMENTS OF THE INVENTION

Reference will now be made in more detail to presently preferred embodiments of the invention. While the invention is described more fully with reference to these examples, the invention in its broader aspects is not limited to these specific details, representative devices, and illustrative examples as shown and described. The description which follows is to be understood as a broad, teaching disclosure directed to persons of ordinary skill in the art and as not limiting upon the invention.

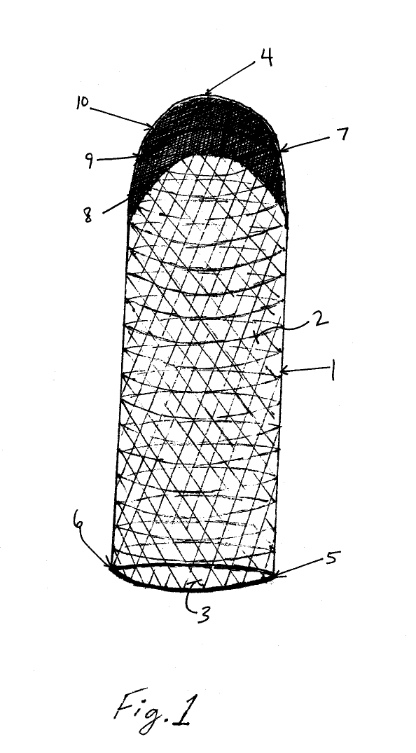

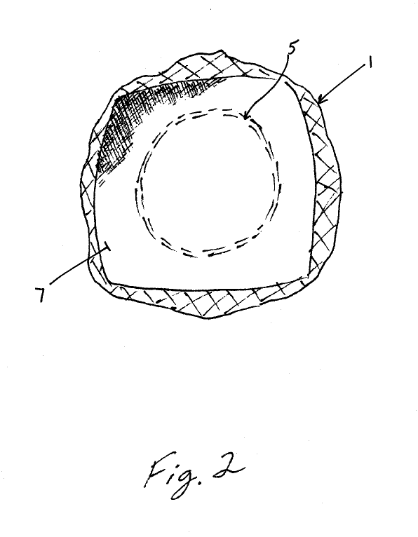

The first preferred embodiment of the invention as shown in FIGS. 1 and 2 is a disposable device that includes a body portion [1] constructed with a light weight breathable mesh netting [2] made from polypropylene, polyester, nylon, cotton, or similar material, accommodating the natural expanding and contracting of the penis, and allowing for breathability against the skin and comfort. The body portion [1] is shaped to go over the penis, with an open end [3] allowing for insertion of the penis, and closed end [4]. The rim of the open end [5] has a soft elastic band [6] allowing the device to gently grip the base of the user's penis preventing the device from falling off. A sheet [7] is affixed to the interior of the closed end [4] of the body and rests against the end of the user's penis while the user is wearing the device. The sheet [7] has a moisture absorbent layer [8], and a layer of water-resistant or waterproof material [9] to prevent leakage onto the user's clothing. The sheet is affixed to the inside of the closed end of the body portion with a thin film of adhesive [10] so that the sheet is comfortably fixed in place against the end of the user's penis when worn. Under this first preferred embodiment of the invention, the device is disposable after soiling.

A second preferred embodiment of the invention includes the same basic structure but with a sheet having an adhesive layer [10] consisting of a non-permanent adhesive material, allowing the user to remove the entire sheet from the body and replacing it with a new sheet. Under this second preferred embodiment of the invention, the flexible mesh netting [2] can be cleaned and re-worn with removable and replaceable sheets.

The device embodied in this invention can be constructed by using various materials known in the art. Upon reading this disclosure, those skilled in the art will appreciate that various changes and modifications may be made to the preferred embodiments of the invention and that such changes and modifications may be made without departing from the spirit of the invention.

What is claimed is as follows:

1. A male urinary incontinence device for absorbing minor urinary leakage or dribbling, the device comprising, in combination:
a body portion being generally cylindrically shaped and having an open end and a closed end on opposing sides of the length, the body portion being symmetrical along the longitudinal axis, the body portion being made of a material that collapses when not in use and when in use expands to the length of the user's penis allowing for the natural expanding and contracting of the penis while the device is being worn, the body portion being constructed of a material that allows for breathability of the skin the closed end of the body portion being of a rounded dome shape to conform to the tip of the penis;
a open end having an elastic band affixed and integrally conformed thereto, the elastic band allowing for the device to be held in place comfortably gripping the skin at the base of the user's penis during use;
a sheet comprised of a layer comprised of a moisture absorbent material, a layer comprised of a moisture impervious material, the sheet being attached to the inside of the closed end of the body portion by adhesive consisting of a non-permanent adhesive material or a permanent adhesive material.
